(19) United States
(12) Patent Application Publication
(10) Pub. No.: US 2005/0039223 A1
(43) Pub. Date: Feb. 17, 2005

(54) Knock-in-mouse

(76) Inventors: Ortrud K. Steinlein, Bonn (DE); Joe Dramiga, Koln (DE)

Correspondence Address: CLARK & ELBING LLP, 101 FEDERAL STREET, BOSTON, MA, 02110, US

(21) Appl. No.: 10/717,373

(22) Filed: Nov. 19, 2003

Related U.S. Application Data
(60) Provisional application No. 60/489,271, filed on Jul. 21, 2003.

(30) Foreign Application Priority Data
Nov. 22, 2002 (DE) DE 102 54 652.5

(51) Int. Cl. A01K 67/027

(52) U.S. Cl. 800/18; 435/320.1

(57) ABSTRACT

The present invention relates to transgenic animals, particularly a knock-in-mouse, as well as to a targeting vector, which is provided for the generation of an animal such as a knock-in-mouse. The present invention further relates to stem cells, preferably murine embryonic stem cells, containing said targeting vector as well as to a screening method for the identification of compounds for the treatment of the human epilepsy syndrome, particularly the familial autosomal dominant nocturnal frontal lobe epilepsy (ADNFLE).

The present invention relates to transgenic animals, particularly to a knock-in mouse and a targeting vector intended for the generation of such an animal like a knock-in mouse. Furthermore, the present invention relates to stem cells, preferably murine embryonic stem cells comprising said targeting vector as well as to a screening method for the identification of compounds for the treatment of human epilepsy syndrome, particularly familiar nocturnal frontal lobe epilepsy (ADNFLE).

According to recent estimations, about 0.4 to 1% of the population suffer from epilepsy, rendering it the second most frequent neurological disease. Though the available antiepileptic drugs are considered effective, sufficient seizure control is impossible with 15 to 20% of the patients; therefore a significant risk of permanent cerebral damages is given. About every 10th heavy status epilepticus, i.e. heavy seizures in rapid sequence, still leads to death today. 40% of the diseases pertain to idiopathic epilepsies (IE), i.e. epilepsies which no specific causes could be found for yet and which, with a prevalence of 0.6%, pertain to the most frequent neurological diseases (Sander, 1996). Concordance rates of 80% with identical twins and 20% with binovular twins verify a predominantly genetic etiology. A 5-10% risk of falling ill with first grade relatives and intra-familiar variability of the IE in childhood and adolescent years indicate an involvement of several genetic factors.

Characteristic for idiopathic autosomal dominant inherited nocturnal frontal lobe epilepsy (ADNFLE), which was only recently described (Scheffer et al., 1994, 1995), are clusters of motor seizures occurring in the non-REM phase of sleep, which usually occur for the first time in the first or second decade of life. The affected patients wake up shortly after falling asleep or very early in the morning displaying non-specific aura phenomena rapidly followed by motor seizures with tonic or hyperkinetic activities. The beginning of a seizure is marked by gasps, grunts or short vocalizations. In most cases consciousness is maintained during the seizures, for which reason they are often misinterpreted as parasomniae, nightmares or hysterical seizures (Scheffer et al. 1994). ADNFLE shows a 70% penetrance and a considerable intra-familiar variation of seriousness (Scheffer et al., 1997). The majority of the affected patients show a normal intellect, a normal EEG diagnosis and no neurological abnormities (Niedermeyer, 1997, Niedermeyer, 1997b, Scheffer et al., 1995). However, in some families there are also often psychomotor development retardations or psychiatric diseases to be found. There are no indications for pathologic changes of certain brain structures. Localization of the epileptogenic zone by surface EEGs is not possible (Haymann et al., 1997). The man/woman proportion of ADNFLE is 7:3 (Provini et al., 1999). Though ADNFLE persists throughout the entire adult life, it can normally be well treated with Carbamazepin (Gambardella et al., 2000, Provini et al., 1999, Scheffer et al., 1995). However, there are also antiepileptic drug resistant cases of ADNFLE (Scheffer et al., 1997).

In 1995 the genetic coupling of ADNFLE with chromosome locus 20q13.2-13.3 could be demonstrated for an Australian family (Phillips et al. 1995). The molecular-genetic pedigree analysis of said ADNFLE family led to the discovery of a missense mutation in the gene for the α4-subunit of the neuronal nicotinic acetylcholine receptor (neuronal nAChR ) (Steinlein et al. 1995). In the case of said missense mutation, a cytosine in nucleotide 743 was replaced by a thymine, which led to the replacement of a serine by a phenylalanine in the second transmembrane domain (TM2) of the α4-subunit of the nAChR (→mutation S248F). The genetic defect responsible for an idiopathic epilepsy could be identified for the first time here. In addition, the Steinlein team found a Norwegian family with ADNFLE (type 1), to whom was introduced an additional leucine behind leucine 259 at the c-terminal end of the TM2 of the α4-subunit of the nAChR by insertion of 3 nucleotides into nucleotide 776 (→L259-260ins mutation; Steinlein et al., 1997).

In Japan another ADNFLE family (type 1) was found having replaced a cytosine by a thymine due to a missense mutation in nucleotide 755, which led to the replacement of serine 252 by leucine in the TM2 of the α4-subunit of the nAChR (→Mutation S252L; Hirose et al., 1999). Two other teams were able to show that for another form of ADNFLE (type 3) two different missense mutations in the gene for the β2-subunit of the nAChR are responsible, which both affected the same amino acid (→Mutation V287L; de Fusco et al., 2000) and (→Mutation V287M; Phillips et al., 2001).

Only few idiopathic epilepsies, like ADNFLE, follow a simple genetic inheritance. These monogenically caused forms of epilepsy are very useful for the exploration of the causes for epileptic seizures due to the direct correlation of genetic impact and phenotype.

Up to now no animal or mouse model, respectively, existed for a human epilepsy syndrome, but merely mouse mutants showing epileptic seizures in combination with an ataxy (Noebels, 1999, Burgess & Noebels, 1999). These mutations are located in genes for subunits of different voltage-gated calcium channels (Noebels, 1999, Burgess & Noebels, 1999). However, the separation of epileptic seizures and epileptic syndromes is decisive for treatment and prognosis. Prognosis will depend on which epilepsy syndrome the relevant patient suffers from and which etiology underlies the case. Correspondingly, therapy recommendations do not relate to individual forms of seizure, but to the epilepsy syndrome.

Therefore, the problem underlying the present invention is to provide a system for the examination of the human epilepsy syndrome. Another problem underlying the present invention is to provide a screening method for novel drug substances for the treatment of epilepsy.

This problem is solved by the subject matter as characterized in the independent claims and in the description. Further and preferred embodiments of the invention are to be found in the dependent claims and in the description.

In accordance with the invention, the solution to the above-mentioned problem is provided by generation of an animal, preferably a knock-in mouse, having a missense mutation in the β2-subunit of the neuronal nAChR.

Neuronal nAChRs consist of α- and β-subunits in a 2:3 stoichiometry. There are 9 different neuronal α-subunits (α₂-α₁₀) and 3 neuronal β-subunits (β₂-β₄). The ion channels assembled of different subunits differ in their electrophysiological and pharmacological properties. All subunits have 4 transmembrane helices (TM1 to TM4), the inner wall of the pore is probably formed by the TM2 helix. For more detailed information on the neuronal nAChR please see Changeux & Edelstein, Current Opinion in Neurobiology, 2001, 11, 369-377.

The invention is based on the surprising finding that in spite of genetic heterogeneity all mutations coupled with ADNFLE that have been found so far are located in the genes for the subunits of the nAChR. Significant changes particularly relate to the TM2 of the α4- and the β2-subunit, which is part of the channel-forming pore and is responsible for ion selectivity. The functional importance of this domain shows itself inter alia in the fact, that its amino acid composition is strictly conserved when comparing very dissimilar species, e.g. mouse, human, rat.

According to a preferred embodiment, these are the missense mutations V287L or V287M in the gene for the β2-subunit. The specific missense mutation V287L and V287M found in an ADNFLE family are already known, as mentioned at the beginning (de Fusco et al., 2000, Phillips et al., 2001, respectively, see supra).

A murine β2-subunit, i.e. the mus musculus neuronal nicotinic acetlycholine receptor beta 2 (acrb2) gene, is deposited with Gene-Bank under the number AF077187 and publicly available since 1999.

Furthermore, the present invention also relates to further knock-in mice with missense mutations in both the TM2 and the TM3 of other subunits, e.g. the remaining β- and also the different α-subunits, like especially the mutations S248F, 259-260ins mutation, S252L, mentioned at the beginning as well as 766ins3 in the α4-subunit of the nAChr receptor.

Furthermore, the invention particularly relates to a hitherto unknown mutation in the α4-subunit, namely T265I. This means that theronine is replaced by isoleucine in the α4-subunit of the nAChr receptor at position 265. The mutation occurs at the extracellular end of the second transmembrane domain. Functional studies of α4-T265I showed an increased ACh-sensitivity of the mutated receptors. α4-T265I is connected with an unusually low penetrance for epilepsy and with a non-increased Carbamazepin-sensitivity.

According to a preferred embodiment, the knock-in mouse contains the above-mentioned missense mutations homozygous or heterozygous.

The present invention further encompasses a targeting vector containing the following components in functional combination:
the genomic and/or cDNA sequence encoding a subunit of the preferably human or murine nicotinic acetylcholine receptor (nAChr) having a missense mutation in the α4- or β2-subunit, or a part thereof, with said part at least comprising the missense mutation in the α4- or β2-subunit,
a selectable marker gene, and
optionally two recognition sequences for a recombinase flanking the marker gene.
   Preferably the selectable marker is an antibiotic resistance gene.

However, in the present invention also further selection markers can be used, i.e. genes that, under certain selective conditions, only allow the carrier of this group to survive.

The two recognition sequences for a recombinase flanking the marker gene are preferably loxP.

The Cre recombinase stems from the E. coli bacteriophage P1 and mediates the site-specific recombination between two identical loxp motives in an intramolecular or intermolecular manner. The recombinase Cre of the E. coli bacteriophage P1 is a site-specific recombinase mediating a DNA reorganization via its DNA target sequence, namely loxP. The loxP sequences consist of an 8 bp spacer region flanked by two 13 bp inverted repeats, which serve as recognition sequences for the DNA binding of Cre. The recombination event is only dependent on these two components and is conducted with absolute reliability. It was appreciated that the Cre-loxP system, in the same way as the Flp-FRT system of S. cerevisiae, effectively catalyses recombination events in prokaryotic as well as in eukaryotic cells including those from yeast, plants, insects and mammals. Site-specific recombination systems are used to a large extent as tools for conditional genetic changes in single cells and animals. In the case of an excision the region of a DNA sequence between two loxP recognition sequences is cut out.

There is a multiplicity of further site-specific recombination systems based on a two-component system that may be used in the present case. All such systems have in common specific repeating DNA sequences. Each of these sequences consists of two recognition sequences that are separated by a spacer and are inversely repetitive to each other. The two components are identical in this case. Besides the examples already mentioned above, there are still the zygosaccharomyces rouxii pSR1, the resolvase-rfsF and the phage Mu Gin recombinase system to be named.

The present invention further relates to stem cells, preferably murine embryonic stem cells, that have been transfected with a vector of the invention.

The animals provided in the present invention, specifically knock-in mice, are preferably usable in screening methods for the identification of compounds for the treatment of the human epilepsy syndrome, particularly of the familiar nocturnal front lobe epilepsy (ADNFLE) comprising the following steps:
a) providing an animal of the invention, particularly knock-in mouse, and of test compounds;
b) administration of the test compounds to the animal, e.g. the mouse,
c) selection of a test compound leading to alleviation or elimination of the symptoms of the epilepsy syndrome in the mouse, and
d) optionally repeating steps a) to c) with an appropriately modified form of the test compound selected in c).

As test compounds for the use in step a) preferably substances from one of the following groups of substances are used: barbiturates, hydantoines, oxazolidindiones and succinimides. In general, all compounds are to be seen as possible test compounds, which have the following grouping as a common structural element:

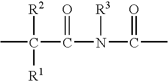

Compounds deviating herefrom can also be used as test compounds, particularly the derivatives of benzodiazepines, sultiam, Carbamazepin and valproic acid.

In one aspect, the present invention relates to compounds for the treatment of the human epilepsy syndrome, particularly of ADNFLE, which have been identified by the screening method mentioned above.

Furthermore, the present invention generally relates to nucleic acid molecules of at least 15 nucleotides in length, which are derived from the above-mentioned alleles of the α4- and the β2-subunits, respectively, and which have a mutation at one of the above-described positions, preferably a missense mutation or equivalent mutations that lead to the same result. The nucleic acid molecules of the invention preferably consist of 15 to 100 nucleotides, more preferably of 15 to 50 nucleotides and most preferably of 15 to 25 nucleotides. Of course, the nucleic acid molecules of the invention may comprise further nucleic acid sequences, e.g. in the form of fusion polynucleotides, which, in certain embodiments, again can encode fusion proteins. Of course, the present invention also relates to partial and complete cDNA molecules which encode the variants of the α4- and the β2-subunit of the nACh receptor as described above and in the Examples.

Furthermore, the invention relates to binding molecules, particularly antibodies and fragments and derivatives thereof, which are binding to the α4- or the β2-subunit of the nACh receptor, respectively, and are specific for the above-described missense mutations, so that the binding molecules of the invention may be applied as targeted diagnostics and, if desired, therapeutics.

Furthermore, the invention relates to nucleic acid molecules, which encode said binding molecules, particularly antibody sequences, as well as vectors that encode one of the above-described nucleic acid molecules and sequences, and host cells comprising said nucleic acid molecules or vectors, respectively. Particularly preferred in this case are animal host cells, in which one or both of the subunits of the nACh receptor of the invention are expressed, and which reconstitute a more or less functional receptor, see also the Examples.

Accordingly, the present invention also generally relates to screening methods using nucleic acid molecules of the invention, proteins encoded by said nucleic acid molecules and the described host cells for the discovery of specific agents. General screening methods that are applied in accordance with the invention are known to the person skilled in the art from the prior art. For example, the expression system as described in the Examples may also be used for a screening method according to the invention.

In addition, the present invention relates to diagnostic compositions comprising one of the above-described components like nucleic acid molecules, peptides that are derived from the α4- or β2-subunit of the nACh receptor and carrying the respective missense mutation, specific antibodies for said peptides, vectors and/or host cells, and which are preferably usable for diagnostic and screening methods. Examples are pharmacogenomic and forensic uses, SNP analysis, drug tailoring and others.

Likewise, the present invention relates to compositions, preferably pharmaceutical compositions, comprising one of the above-described components, particularly specific antibodies and/or agents discovered by the screening method of the invention, which are ideally capable of either compensating the described mutation and/or of influencing the nACh receptor inasmuch that it basically functions normally again despite the respective mutation.

According to the present description of the invention it is clear to the person skilled in the art that the invention is not limited to e.g. mouse, but may be used generally, which also applies to embodiments described otherwise, like the knock-in mouse. Other animals that can be generated according to the invention are e.g. hamsters, rabbits, rats etc. Accordingly, the present invention generally relates to transgenic animals having a missense mutation in the α4- or β2-subunit of the neuronal nicotinic acetylcholine receptor (nAChR) as well as generally to stem cells of mammals that have been genetically engineered according to the embodiment described above.

Concerning diagnosis and screening methods, the present invention also relates to uses applying nucleic acid molecules, proteins or peptides, respectively, antibodies or host cells of the invention. In this respect, the present invention particularly relates to chips and arrays, particularly micro arrays, which are loaded with one of the above-described components of the invention, and which may, if necessary, contain further components like nucleic acids of other alleles of the subunits of the nACh receptor.

In a further aspect, the present invention relates to a pharmaceutical composition having one or more of the compounds identified as described above and a pharmaceutically acceptable carrier.

The agents of the present invention are preferably mixed with appropriate carriers or carrier substances in doses that will treat or at least alleviate the disease. Such a composition may (in addition to the agents and the carrier) comprise diluents, fillers, salts, buffers, stabilizers, dilution mediators and other substances that are per se already known. The term “pharmaceutically acceptable” is meant to define a non-toxic substance that does not disturb the effectiveness of the biological activity of the active ingredient or agent, respectively. Selection of the carrier depends on the way of administration.

The pharmaceutical composition may additionally contain further substances, which increase the effectiveness of the agent or which complement its activity or its use in treatment. Such additional factors and/or substances may be comprised in the pharmaceutical composition to achieve a synergistic effect or to minimize side effects or undesired effects, respectively. Techniques for the formulation or preparation and administration, respectively, of compounds of the invention are to be found in “Remington's Pharmaceutical Sciences”, Mack Publishing Co., Easton, Pa., latest edition.

The term “therapeutically effective dose” relates to the amount of the compound that is sufficient to achieve improvement of the symptoms, e.g. treatment, cure, prevention or improvement of such conditions. Appropriate ways of administration may include e.g. oral, rectal, transmucosal, intestinal or parenteral administration, comprising intramuscular, subcutaneous, intramedullary injections as well as intrathecal, directly intraventricular, intravenous, intraperitoneal or intranasal injections.

The orally administered pharmaceutical preparations include hard as well as soft gelatin capsules, which are manufactured using gelatin and a softener, like e.g. glycerol or sorbitol. The hard gelatin capsules may contain effective compounds mixed with fillers, like e.g. lactose, binding agents, like e.g. starches, and/or lubricants, like e.g. talcum or magnesium stearate, and optionally stabilizers. In the case of soft gelatin capsules, the effective compounds are preferably dissolved or suspended in appropriate liquids, like e.g. buffered salt solution. Stabilizers may additionally be used.

In addition to administration in liquid form, e.g. in a gelatin capsule or another suitable carrier, the pharmaceutical preparations can contain suitable carrier substances in order to facilitate the processibility of the effective compounds in the preparation, which are used pharmaceutically. Therefore, pharmaceutical preparations for oral administration can be obtained by binding the solution of the effective compounds to a solid carrier, optionally grinding of the resulting mixture and processing the mixture to granulates after adding of suitable auxiliary substances, if desired or necessary, for the production of tablets or tablet cores.

Suitable carrier substances are in particular fillers, like e.g. sugar, e.g. lactose, sucrose, mannitol or sorbitol, cellulose preparations and/or calcium phosphate, e.g. tricalcium phosphate or calcium hydrogen phosphate as well as binding agents like e.g. starch, e.g. corn starch, wheat starch, rice starch, potato starch, gelatin, traganth, methylcellulose, hydroxypropylmethylcellulose, sodium carboxymethyl-cellulose and/or polyvinylpyrrolidone. If desired, sprinkling or disintegrating substances, respectively, can be added, like e.g. the above-mentioned starches and also carboxymethyl starch, cross-linked polyvinylpyrrolidone, agar or alginic acid or salts thereof like e.g. sodium alginate. Auxiliary substances are, in particular, flow-regulating substances and lubricants, e.g. silica, talcum, stearic acid and salts thereof, like e.g. magnesium stearate or calcium stearate and/or macrogol. Tablet cores are equipped with suitable coating. If necessary, they are resistant to gastric juices. For this purpose, concentrated sugar solutions can be used optionally containing Gummi arabicum, talcum, polyvinylpyrrolidone, macrogol and/or titaniumdioxide, varnish solutions and suitable organic solvents or solvent mixtures. For the generation of coatings or protective layers which are resistant to gastric acid, solutions of suitable cellulose preparations, like e.g. acetylcellulosephthalate or hydroxypropylmethyl cellulosephthalate, are used.

Suitable preparations for intravenous or parenteral administration include aqueous solutions of the effective compounds. A typical composition for an intravenous infusion can be produced to contain e.g. 250 ml sterile Ringer's solution and e.g. 10 mg drug substance. See Remington's Pharmaceutical Science (15th edition, Mack Publishing Company, Easton, Pa., 1980). In addition, suspensions of the active compounds, like e.g. oily injection suspensions, can be administered. Aqueous injection suspensions can contain substances that increase the viscosity of the suspension and include e.g. sodium carboxymethylcellulose, sorbitol and/or dextran. Optionally, the suspension can further contain stabilizers.

The above-mentioned compounds or the pharmaceutical composition, respectively, are preferably used for the treatment of the human epilepsy syndrome, particularly of the ADNFLE.

The disclosure content of the above- and below-cited documents from the prior art is hereby incorporated in this application by reference, in particular relating to the production of nucleic acid molecules of the invention, chips and arrays loaded herewith, vectors, host cells, antibodies, transgenic animals etc. This and other embodiments are disclosed and apparent to the person skilled in the art and are comprised by the description and the Examples of the present invention. More detailed literature on one of the above-mentioned materials and electronic tools, which can be used in the sense of the present invention, can be taken from the prior art, e.g. from public libraries using e.g. electronic tools. Furthermore, there are other public databases offering their services, like “medline”, that are available in the internet.

Techniques for carrying out the invention are known to the person skilled in the art and can be taken from the appropriate literature, see e.g. Molecular Cloning A Laboratory Manual, 2nd Ed., ed. von Sambrook, Fritsch und Maniatis (Cold Spring Harbor Laboratory Press: 1989); DNA Cloning, Bande I und II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller und M. P. Calos eds., 1987, Cold Spring Harbor Laboratory); Methods In Enzymology, Vols. 154 and 155 (Wu et al. eds.), Immunochemical Methods In Cell And Molecular Biology (Mayer und Walker, eds., Academic Press, London, 1987); Handbook Of Experimental Immunology, Bande I-IV (D. M. Weir and C. C. Blackwell, eds., 1986); Extracting information from cDNA arrays, Herzel et al., CHAOS 11, (2001), 98-107.

The present invention is illustrated with the following figures as well as with the following Examples. The figures show:

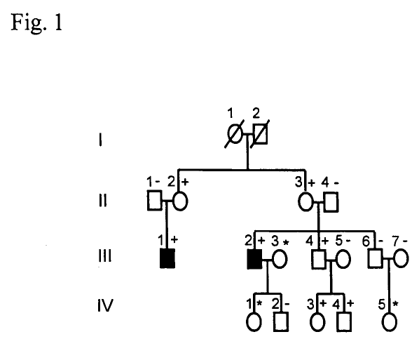

FIG. 1: Shows the pedigree of a German family with ADNFLE (see Example 2); filled symbols indicate the family members with ADNFLE diagnosis. + indicates individuals that were heterozygous concerning the α4-T265I-mutation; − indicates family members without α4-T265I-mutation; * means no molecular analysis available.

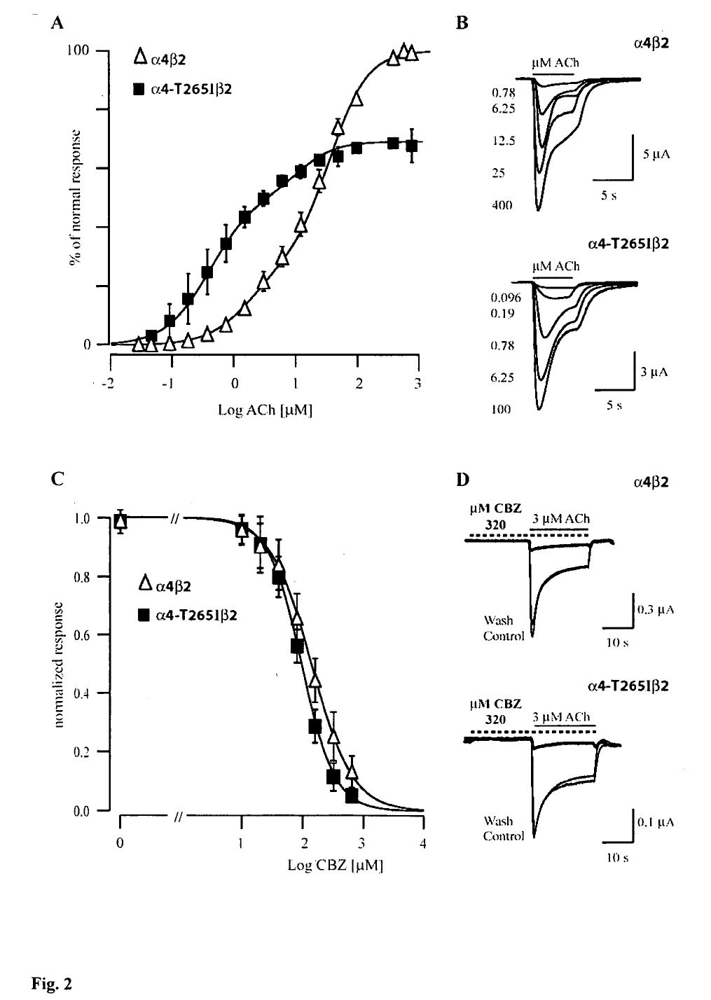

FIG. 2: A, ACh-dose effect curve of control (open triangles) and of receptors containing α4-T265I (filled triangles). The curves passing through the triangles are the best fits achieved with equation 1.
B, ACh-evoked currents overlap for control (upper traces) and for the mutant containing a receptor (lower traces). The bars indicate the time course of ACh administration.
C, CBZ dose-effect inhibition curves for control (open triangles) and for receptors containing α4-T265I (filled squares). The data points were measured at the end of ACh- and CBZ-co-administration. The curves passing through the data points are the best fits achieved with equation 2.
D, ACh-evoked currents, which were first recorded in control (thin line) and then in the presence of CBZ (thick line), overlap. The upper traces were recorded in an oocyte expressing the α4-T265I mutant.

### EXAMPLES

In the following, the present invention is illustrated by way of an example. However, the invention is not limited to this concrete example.

### Example 1

First the gene for the β2-subunit of the nACHR from a genomic bank as well as the corresponding cDNA of the mouse stem C57/BL6 was isolated, subcloned and sequenced from both sides. The point mutation (ADNFLE missense mutation V287L) was introduced by means of PCR into both the genomic DNA and the cDNA of the gene for the β2-subunit of the mouse.

The genomic DNA was cloned into a flox-and-replace-vector for the transfection of ES cells. Isogenic embryonic ES cells of the mouse strain C57/BL6 and feeder cells (murine embryonic fibroblasts) of the same strain are already being cultivated by the inventor in another project. With the help of the Cre-plasmid for the transient expression of Cre in ES cells, the neo cassette can be removed from the genome of the transfected ES cells after transfection.

### Transfection of the ES Cells

First the gene in murine embryonic stem cells (ES cells) of the C57/BL6 strain is replaced via homologous recombination by its mutated counterpart, this is done by means of a so-called targeting vector. Beside the mutated gene, our targeting vector contains selectable markers, namely genes for antibiotic drug resistances. The targeting vector is brought into the ES cells by electroporation. Hereby, a skillful combination of selection conditions also facilitates the targeted isolation of only those ES cells, which have the modified gene incorporated at the correct (homologous) position and not accidentally somewhere in the genome (positive/negative selection). We will then investigate DNA samples of each isolated clone by means of Southern blot hybridization and PCR in order to find out those transfectants that have the transgene incorporated at the correct position in the genome.

### Deletion of the Selection Marker Cassette

Those transfectants having incorporated the transgene at the correct position in the genome are now further cultivated and transfected with a Cre plasmid. This facilitates the deletion of the selection cassette from the genome of the ES cells by means of transient Cre expression.

The properties of the recombination systems, e.g. of the Cre system, were combined with different conventional strategies for gene targeting and displacement (4, 5). Usually, conventional genomic changes are based on the targeted integration of a modified allele. In eucaryotic and prokaryotic cells the integration event is achieved by homologous recombination within the flanking regions of the allele in question. A positive genetic marker is obligatory for the selection of homologous recombination events, which occur very rarely in most genetic systems. Therefore, it is often desirable to delete this marker in a subsequent step, preferably in connection with the remaining wild type allele. For the deletion of the marker gene (or of DNA segments that are to be deleted), incorporated loxP sequences facilitate the efficient excision of the loxP-flanked DNA segment in a strictly Cre-dependent manner. The cut out fragment is circularized and lost by degradation, however, one single loxP sequence remains in the modified gene locus.

### Blastocyst Injection

Subsequently, the ES cells containing the floxed allele at the correct position and having the selection cassette deleted have to be injected into the blastocyst in order to obtain chimerae. (3 to 4 days after fertilization). The blastocyst containing about 100 cells is implanted in pseudo-pregnant females. Embryonic stem cells of the mouse are capable of participating in all aspects of the development including the germ track. Therefore, the injected ES cells can become germ cells, which then transmit the mutated gene. The developing mice are chimerae with respect to the changed gene, hence some of the tissues derive themselves from the injected ES cells, others from the normal blastocyst.

The chimerae can be identified especially easily if the mouse strain donating the blastocyst is white and the mouse strain, which the ES cells come from, has a dark fur color. The chimeric descendants then show a spotted fur. We use the white CD 20 strain, a Balb/c strain 20 times re-crossed in C57/BL6, as blastocyst donor.

### Generation of Heterozygous Mice

In most cases also the germ cells are chimeric, however, due to the fact that they are haploid, only some of them contain the variant with the mutated gene. Test hybridizations will now be performed in order to examine whether the introduced ES cells have become germ cells. The resulting chimeric males are hybridized with wild type females of the strain C57/BL6 in order to generate heterozygous mutants. The mutants can be unambiguously identified by means of Southern blot and PCR.

### Generation of Homozygous Mice

The heterozygous F1 mutants are crossed among each other, which produces homozygous mutants in the F2 generation. The mutants can be unambiguously identified by means of Southern blot and PCR.

### Example 2

In Example 2 it is described how the hitherto unknown mutation in the α4-subunit, namely T265I, was identified and how its pharmacological properties were characterized by reconstitution experiments in Xenopus oocytes.

### Patients

The pedigree of a German family with 16 living members (including 5 spouses) is shown in FIG. 1A. Clinical data were obtained from all living members. Two individuals (III1, III2), aged 31 and 38 years, were affected. They underwent clinical investigations, namely interictal EEGs (FIG. 1B) and cranial MRI scans. EEGs were also obtained from the 5 unaffected carriers of the CHRNA4 mutation (mutation T265I in the α4-subunit). A total of 9 family members and 4 spouses were screened for the presence or absence of the mutation. The control sample consisted of DNA from 79 German unrelated healthy individuals.

### Mutation Screening and Verification of the Mutation

A mutation screening was carried out as previously described (1), but direct sequencing was used instead of single strand conformation analysis (SSCA). For verification of the mutation, for analysis of the complete ADNFLE family and for screening the control sample a PCR restriction fragment length assay was developed using the primer n24942 (5′ GGCGAGTGGGTCATCGTGG) and N54624 (5′ GCTCGGGCCAGAAGCGCGG). A standard PCR was performed using a buffer containing 2.0 mM MgCl₂ and 5% DMSO; an annealing temperature of 64.3° C. and an extension time of 45 seconds were chosen. 5 μl of the resulting PCR product were treated with 4 U TaqI (Roche Molecular Biochemicals) for 7.5 hours at 65° C. Subsequently, 7 μl of the restricted PCR product were run on a 10% polyacrylamide gel (15 V/cm) for 2 to 3 hours. After the electrophoresis the bands were visualized using a standard silver staining protocol. The TagI restriction digest yielded the following pattern: wild type allele 42bp+497 bp; mutated allele, 42 bp+260+237 bp (FIG. 2A).

### CHRNA4 Mutagenesis

The expression vector pSV2-ZEO (Invitrogen) containing the CHRNA4 wild type sequence was used for in vitro mutagenesis. The expression clone CHRNA4-T265I was constructed with the QuikChange Mutagenesis-Kit (Stratagene) according to the manufacturer's instructions using the following primers:

nl554 5′-

GTCTTCCTGCTGCTCATCATCGAGATCATCCCGTCCACC and n1594, 5′-

GGTGGACGGGATGATCTCGATGATGAGCAGCAGGAAGAC. Single colonies were screened by the TagI restriction assay described above and positive clones were sequenced for verification.

### Screening of Different nAChR Subunits

The TM1 to TM3-containing part of the nAChR subunit genes CHRNA2-CHRNA3, CHRNA5-CHRNA7, CHRNA9-CHRNA10 and CHRNB2-CHRNB4 were amplified by PCR and subsequently screened by direct sequencing in the family members II2 and III1.

### Elektrophysiology

Expression experiments in Xenopus oocytes were conducted as previously described (2). Nuclear cDNA injections were conducted using a ratio of 0.5 control α4, 0.5 mutated α4 and 1 β2 (total amount 2 ng/oocytes) (3). The oocytes were kept in BARTH solution containing 88 mM NaCl, 1 mM KCl, 2.4 mM NaHCO₃, 10 mM HEPES, 0.82 mM MgSO₄7H₂O, 0.33 mM Ca(NO₃)₂.4H₂O, 0.41 mM CaCl₂.6H₂O, pH 7.4, adjusted with NaOH and supplemented with kanamycin (20 μg/ml), penicillin (100 μg/ml) and streptomycin (100 μg/ml). Recordings were made 2 to 3 days later using the two electrode voltage clamp technique with a GENE-CLAMP amplifier (Axon Instrument). During the experiments the oocytes were continuously rinsed with OR2 solution: 82.5 mM NaCl, 2.5 mM KCl, 2.5 mM CaCl₂, 5 mM HEPES, pH 7.4, adjusted with NaOH. Unless stated otherwise, the holding potential was −100 mV and the experiments were performed at 18° C. ACh dose response curves were best fitted by the sum of two empirical Hill equations: y=Imax{a/(1+(EC₅₀H/x)^{nH1})+(1−a)/(1+(EC₅₀L/x)^{nH2})} (equation (1)). Imax is the maximal current amplitude and x is the agonist concentration. EC₅₀H, nH1 and a are the half-effective concentration, the Hill coefficient the percentage of receptors in high-affinity state, respectively, whereas EC₅₀L and nH2 correspond to the half-effective concentration and the Hill coefficient in low-affinity state. Carbamazepine (CBZ) inhibition curves were fitted using a similar equation: y=1/(1+(x/IC₅₀)^{nH}) (equation (2)), where x is the antagonist concentration. IC₅₀ and nH are the half-inhibition concentration and the Hill coefficient, respectively.

The new CHRNA4 mutation α4-T265I causes nocturnal frontal lobe epilepsy (ADNFLE). In comparison to all cAChR mutations known hitherto, the α4-T265I mutation shows a remarkably low penetrance, which is consistent with a main gene effect, but not with a monogenic inheritance. Our results show that a continuum between monogenic and oligogenic forms of nocturnal frontal lobe epilepsy exists. Former mutation studies concentrated on typical ADNFLE families, in which the existence of many affected family members in the subsequent generations suited an autosomal-dominant inheritance model. Therefore, it was not likely that they discovered main gene effects. It would be interesting to screen patients with apparent sporadic forms of nocturnal frontal lobe epilepsy in order to estimate the frequency of low-penetrance CHRNA4 or CHRNB2 mutations in this group.

### LITERATURE

### Review Articles

Steinlein, O. (1996): Familiäre nächtliche Frontallappenepilepsie. Nervenarzt 67:870-874
Steinlein, O. (1999): Die Genetik der idiopathischen Epilepsien. Deutsches Ärzteblatt 96:1047-1052
Steinlein, O. (1999): Idiopathic epilepsies with a monogenic mode of inheritance. Epilepsia 40 Suppl 3:9-11
Epilepsia 2002;43 Suppl 5:112-22
Neurophysiol Clin 2002 April;32(2):99-107
Epilepsia 2002 April;43(4):362-4
Pflugers Arch 2001 August;442(5):642-51

### Original Articles

De Fusco, M.; Becchetti, A.; Patrignani, A.; Annesi, G.; Gambardella, A.; Quattrone, A.; Ballabio, A.; Wanke, E.; Casari, G. (2000): The nicotinic receptor beta-2 subunit is mutant in nocturnal frontal lobe epilepsy. Nature Genet. 26: 275-276.
Haymann, M.; Scheffer, I. E.; Chinvarun, Y.; Berlangieri, S. U.; Berkovic, S. F. (1997): Autosomal dominant nocturnal frontal lobe epilepsy: demonstration of focal frontal onset and intrafamilial variation. Neurology 49: 969-975.
Hirose S, IwataH, Akiyoshi H, Kobayashi K, Ito M, Wada K, Kaneko S, Mitsudome A. (1999): A novel mutation of CHRNA4 responsible for autosomal dominant nocturnal frontal lobe epilepsy. Neurology 53: 1749-1753.
Niedermeyer (1987) Maturation of the EEG: Development of waking and sleep patterns. In: Niedermeyer E, Lopes da Silva F (eds) Electroencephalogy. Urban and Schwarzenberg, Baltimore, 133-157.
Niedermeyer E (1987) Sleep and EEG. In: Niedermeyer E, Lopes da Silva F (eds) Electroencephalogy. Urban and Schwarzenberg, Baltimore, 119-132.
Phillips, H. A.; Favre, I.; Kirkpatrick, M.; Zuberi, S. M.; Goudie, D.; Heron, S. E.; Scheffer, I. E.; Sutherland, G. R.; Berkovic, S. F.; Bertrand, D.; Mulley, J. C. (2001): CHRNB2 is the second acetylcholine receptor subunit associated with autosomal dominant nocturnal frontal lobe epilepsy. Am. J. Hum. Genet. 68: 225-231
Phillips, H. A.; Scheffer, I. E.; Berkovic, S. F.; Hollway, G. E.; Sutherland, G. R.; Mulley, J. C. (1995): Localization of a gene for autosomal dominant nocturnal frontal lobe epilepsy to chromosome 20q 13.2. Nature Genet. 10: 117-118.
Sander T (1996): Mol Med Today 2: 173-80
Scheffer, I. E.; Bhatia, K. P.; Lopes-Cendes, I.; Fish, D. R.; Marsden, C. D.; Andermann, E.; Andermann, F.; Desbiens, R.; Keene, D.; Cendes, F.; Manson, J. I.; Constantinou, J. E. C.; McIntosh, A.; Berkovic, S. F. (1995): Autosomal dominant nocturnal frontal epilepsy: a distinctive clinical disorder. Brain 118: 61-73.
Scheffer, I. E.; Hopkins, I. J.; Harvey, A. S.; Berkovic, S. F. (1994): New autosomal-dominant partial epilepsy syndrome. (Abstract) Pediat. Neurol. 11: 95.
Steinlein, O. K.; Magnusson, A.; Stoodt, J.; Bertrand, S.; Weiland, S.; Berkovic, S. F.; Nakken, K. O.; Propping, P.; Bertrand, D. (1997): An insertion mutation of the CHRNA4 gene in a family with autosomal dominant nocturnal frontal lobe epilepsy. Hum. Molec. Genet. 6: 943-947.
Steinlein, O. K.; Mulley, J. C.; Propping, P.; Wallace, R. H.; Phillips, H. A.; Sutherland, G. R.; Scheffer, I. E.; Berkovic, S. F. (1995): A missense mutation in the neuronal nicotinic acetylcholine receptor alpha-4 subunit is associated with autosomal dominant nocturnal frontal lobe epilepsy. Nature Genet. 11: 201-203.
Steinlein, O., Noebels, J. (2000): Ion channels and epilepsy in man and mouse. Current Opinion in Genetics & Development 10: 286-291

### Literature for Example 2

1. Steinlein O, Weiland S, Stoodt J, Propping P. Exon-intron structure of the human neuronal nicotinic acetylcholine receptor alpha 4 subunit (CHRNA4). Genomics 1996;32:289-294.
2. Bertrand D, Cooper E, Valera S, Rungger D, Ballivet M. Electrophysiology of neuronal nicotinic acetylcholine receptors expressed in Xenopus oocytes following nuclear injection of genes or cDNA. In: Methods in Neuroscience (Conn M, ed) 1991, pp 174-193. New York: Academic Press.
3. Moulard B, Picard F, le Hellard S, et al. Ion channel variation causes epilepsies. Brain Res Brain Res Rev 2001;36:275-284.

1. A transgenic non-human animal, comprising a missense mutation in the α4- or β2-subunit of the neuronal nicotinic acetylcholine receptor (nAChr).

2. The animal of claim 1, wherein said missense mutation is V287L or V287M in the gene encoding said β2-subunit of said nAChr receptor.

3. The animal of claim 1, wherein said missense mutation is selected from the group consisting of 259-260ins, S252L, 766ins3 and T265I and said mutation is in said α4-subunit of said nAChr receptor.

4. The animal of claim 1, wherein said animal is homozygous for said missense mutation.

5. The animal of claim 1, wherein said animal is heterozygous for said missense mutation.

6. A targeting vector comprising a nucleic acid sequence encoding a subunit of a human or murine, nicotinic acetylcholine receptor (nAChr) having a missense mutation in the α4- or β2-subunit, or a part of said subunit, wherein said part comprises at least said missense mutation in the α4- or β2-subunit operably linked to a selectable marker gene.

7. The targeting vector of claim 6, wherein the selectable marker is an antibiotic resistance gene.

8. The targeting vector of claim 6, wherein said vector further comprises two recognition sequences for a recombinase that, flank the marker gene.

9. The targeting vector of claim 6, wherein the β2-subunit comprises a V287L or V287M missense mutation.

10. The targeting vector of claim 6, wherein the α4-subunit has a missense mutation selected from the group consisting of 259-260ins, S252L, 766ins3 or T265I.

11. A stem cell, comprising a vector of claim 6.

12. A screening method for the identification of compounds for the treatment of the human epilepsy syndrome, comprising the following steps:
a) providing an animal of claim 1 and a test compound,
b) administering said test compound to said animal,
c) selecting a test compound that alleviates or eliminates symptoms of an epilepsy syndrome in said animal.

13. The screening method of claim 12, wherein said test compound is selected from the group consisting of barbiturates, oxazolidindiones, succinimides, derivatives of benzodiazepines, sultiam, Carbamazepin, valproic acid and compounds comprising formula I: 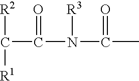
wherein R¹ and R² are alkyl or aryl residues and R³ is H or an alkyl residue.

14. A compound for the treatment of a human epilepsy syndrome, identified according to the method of claim 12.

15. A pharmaceutical composition comprising a therapeutically effective dose of a compound identified according to the method of claim 12 in a pharmaceutically acceptable carrier.

16. A method for treating a human epilepsy syndrome, by administering to a patient a composition identified according to the method of claim 15.

17. The transgenic non-human animal of claim 1, wherein said animal is a knock-in mouse.

18. The targeting vector of claim 6, wherein said nucleic acid is a genomic and/or cDNA nucleic acid sequence.

19. The screening method of claim 12, where in said human epilepsy syndrome is familial autosomal dominant nocturnal frontal lobe epilepsy (ADNFLE).

20. The screening method of claim 12, wherein said method further comprises:
d) repeating steps a) to c) with a modified form of the test compound chosen in c).


SEQUENCE LISTING (4 sequences)

SEQ ID NO: 1 - DNA, 19 bp, Artificial Sequence
  Primer
ggcgagtgggtcatcgtgg

SEQ ID NO: 2 - DNA, 19 bp, Artificial Sequence
  Primer
gctcgggccagaagcgcgg

SEQ ID NO: 3 - DNA, 39 bp, Artificial Sequence
  Primer
gtcttcctgctgctcatcatcgagatcatcccgtccacc

SEQ ID NO: 4 - DNA, 39 bp, Artificial Sequence
  Primer
ggtggacgggatgatctcgatgatgagcagcaggaagac